(19) United States
(12) Patent Application Publication
(10) Pub. No.: US 2025/0224390 A1
(43) Pub. Date: Jul. 10, 2025

(54) DEVICE AND METHOD WITH CELL ELECTRICAL SIGNAL MEASUREMENT

(71) Applicant: Samsung Electronics Co., Ltd., Suwon-si (KR)

(72) Inventors: Jaechun LEE, Suwon-si (KR); Chisung BAE, Suwon-si (KR)

(73) Assignee: Samsung Electronics Co., Ltd., Suwon-si (KR)

(21) Appl. No.: 18/958,830

(22) Filed: Nov. 25, 2024

(30) Foreign Application Priority Data
Jan. 9, 2024 (KR) 10-2024-0003719

(51) Int. Cl. G01N 33/483; C12M 1/34; G01N 27/02

(52) U.S. Cl. CPC G01N 33/4836; C12M 41/46; G01N 27/02

(57) ABSTRACT

An electronic device includes a plurality of sensing electrodes in contact with a medium for culturing cells, a reference electrode connected to a ground and in contact with the medium, a voltage generator connected to the reference electrode, a plurality of electric current generators, each of which is connected to each of the plurality of sensing electrodes and configured to apply an electric current to a corresponding one of the sensing electrodes, a plurality of voltage sensors, each of which is connected to each of the plurality of sensing electrodes to measure the voltage of the corresponding sensing electrode, and one or more processors configured to obtain, in response to applying a reference voltage by the voltage generator, a first value output from a target voltage sensor among the plurality of voltage sensors connected to a target sensing electrode among the plurality of sensing electrodes, obtain, in response to applying a reference electric current by a target electric current generator connected to the target sensing electrode among the plurality of electric current generators, a second value output from the target voltage sensor, and determine an impedance of the target sensing electrode based on the reference voltage, the reference electric current, the first value, and the second value.

### CROSS-REFERENCE TO RELATED APPLICATIONS

This application claims the benefit under 35 USC § 119 (a) of Korean Patent Application No. 10-2024-0003719, filed on Jan. 9, 2024 in the Korean Intellectual Property Office, the entire disclosure of which is incorporated herein by reference for all purposes.

### BACKGROUND

### 1. Field

The following description relates to a device and method with cell electrical signal measurement.

### 2. Description of Related Art

A variety of methods may be used to analyze a human biometric signal. For example, signals from neurons measured by a plurality of electrodes may be used to analyze brain waves. In addition, the manner of transmitting signals of a brain may be interpreted by passing an electric current of a certain magnitude through neurons of the brain and observing intracellular signals. A microelectrode array may include a plurality of electrodes, each of which may measure an electrical signal generated at the cellular level.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

In one or more general aspects, an electronic device includes a plurality of sensing electrodes in contact with a medium for culturing cells, a reference electrode connected to a ground and in contact with the medium, a voltage generator connected to the reference electrode, a plurality of electric current generators, each of which is connected to each of the plurality of sensing electrodes and configured to apply an electric current to a corresponding one of the sensing electrodes, a plurality of voltage sensors, each of which is connected to each of the plurality of sensing electrodes to measure the voltage of the corresponding sensing electrode, and one or more processors configured to obtain, in response to applying a reference voltage by the voltage generator, a first value output from a target voltage sensor among the plurality of voltage sensors connected to a target sensing electrode among the plurality of sensing electrodes, obtain, in response to applying a reference electric current by a target electric current generator connected to the target sensing electrode among the plurality of electric current generators, a second value output from the target voltage sensor, and determine an impedance of the target sensing electrode based on the reference voltage, the reference electric current, the first value, and the second value.

For the determining of the impedance of the target sensing electrode, the one or more processors may be configured to determine the impedance of the target sensing electrode from the reference voltage, the reference electric current, the first value, and the second value, regardless of a parasitic element of the target sensing electrode and a gain of the target voltage sensor.

The one or more processors may be configured to obtain a third value output from the target voltage sensor in response to applying the reference electric current by the target electric current generator after the medium is removed, and determine a gain of the target voltage sensor based on the reference voltage, the first value, the second value, and the third value.

The one or more processors may be configured to sense a second reference electric current flowing through the reference electrode in response to applying the reference electric current by the target electric current generator, obtain a fourth value output from the target voltage sensor in response to applying a fourth reference electric current by the target electric current generator after the medium is removed, and determine the impedance of the target sensing electrode based on the reference voltage, the second reference electric current, the first value, the second value, and the fourth value.

The reference electric current applied by the target electric current generator may be determined based on the second reference electric current, the second value, and the fourth value.

In response to applying the reference voltage by the voltage generator, the plurality of electric current generators may not operate, and in response to applying the reference electric current by one of the plurality of electric current generators, other electric current generators than the one and the voltage generator may not operate.

The reference voltage applied by the voltage generator may be an alternating current (AC) voltage, the reference electric current applied by the target electric current generator may be an AC electric current, and the target voltage sensor may be configured to output a digital value amplified according to a predetermined gain of an analog value of a voltage detected at an input terminal of the target voltage sensor.

Each of the plurality of electric current generators and each of the plurality of voltage sensors may be disposed connected to a sensing electrode that corresponds to an inside of the device.

In one or more general aspects, a method of a device includes obtaining, in response to applying a reference voltage by a voltage generator connected to a ground and a reference electrode in contact with a medium for culturing cells, a first value output from a target voltage sensor connected to a target sensing electrode among a plurality of sensing electrodes in contact with the medium, obtaining, in response to applying a reference electric current by a target electric current generator connected to the target sensing electrode, a second value output from the target voltage sensor, and determining an impedance of the target sensing electrode based on the reference voltage, the reference electric current, the first value, and the second value.

The determining of the impedance of the target sensing electrode may include determining the impedance of the target sensing electrode from the reference voltage, the reference electric current, the first value, and the second value, regardless of a parasitic element of the target sensing electrode and a gain of the target voltage sensor.

The method may include obtaining a third value output from the target voltage sensor in response to applying the reference electric current by the target electric current generator after the medium is removed, and determining a gain of the target voltage sensor based on the reference voltage, the first value, the second value, and the third value.

The method may include sensing a second reference electric current flowing through the reference electrode in response to applying the reference electric current by the target electric current generator, obtaining a fourth value output from the target voltage sensor in response to applying a fourth reference electric current by the target electric current generator after the medium is removed, and determining the impedance of the target sensing electrode based on the reference voltage, the second reference electric current, the first value, the second value, and the fourth value.

The reference electric current applied by the target electric current generator may be determined based on the second reference electric current, the second value, and the fourth value.

In response to applying the reference voltage by the voltage generator, a plurality of electric current generators may not operate, and in response to applying the reference electric current by one of the plurality of electric current generators, other electric current generators than the one and the voltage generator may not operate.

The reference voltage applied by the voltage generator may be an alternating current (AC) voltage, the reference electric current applied by the target electric current generator may be an AC electric current, and the target voltage sensor may be configured to output a digital value amplified according to a predetermined gain of an analog value of a voltage detected at an input terminal.

Each of the plurality of electric current generators and each of the plurality of voltage sensors, which are included in the device, may be disposed connected to a sensing electrode that corresponds to an inside of the device.

In one or more general aspects, a non-transitory computer-readable storage medium may store instructions that, when executed by one or more processors, configure the one or more processors to perform any one, any combination, or all of operations and/or methods disclosed herein.

In one or more general aspects, an electronic device includes one or more processors configured to obtain, in response to a reference voltage being applied by a voltage generator connected to a ground and a reference electrode in contact with a medium for culturing cells, a first value output from a voltage sensor connected to a sensing electrode in contact with the medium, obtain, in response to a reference electric current being applied by a electric current generator connected to the sensing electrode, a second value output from the voltage sensor, and determine an impedance of the sensing electrode based on the reference voltage, the reference electric current, the first value, and the second value.

The electric current generator may be in an open state when the first value is output from the voltage sensor, and the voltage generator may be shorted when the second value is output from the voltage sensor.

The one or more processors may be configured to determine a state of a cell of the cells based on the determined impedance.

Other features and aspects will be apparent from the following detailed description, the drawings, and the claims.

### BRIEF DESCRIPTION OF THE DRAWINGS

FIG. 1 illustrates an example of a device for measuring an electrical signal of a cell.

FIG. 2 illustrates an example of components of a device for measuring a potential of a cell.

FIG. 3 illustrates an operation of elements in a device based on a target sensing electrode.

FIG. 4 and FIG. 5 each illustrate an example of an operation of measuring an impedance of a sensing electrode.

FIG. 6 illustrates a flowchart of an example of an operation of measuring an impedance of a sensing electrode.

FIG. 7 illustrates an example of an operation of analog front-end (AFE) calibration.

FIG. 8 illustrates a flowchart of an example of an operation of AFE calibration.

FIG. 9 illustrates an example of an operation of measuring an impedance of a sensing electrode based on a second reference electric current flowing through a reference electrode when a magnitude of a reference electric current generated by an electric current generator is uncertain.

FIG. 10 illustrates a flowchart of an example of an operation of measuring an impedance of a sensing electrode based on a second reference electric current flowing through a reference electrode.

FIG. 11 illustrates an example of a device for measuring an electrical signal of a cell.

FIG. 12 illustrates an example of an operating method of a device for measuring an electrical signal of a cell.

Throughout the drawings and the detailed description, unless otherwise described or provided, the same drawing reference numerals will be understood to refer to the same elements, features, and structures. The drawings may not be to scale, and the relative size, proportions, and depiction of elements in the drawings may be exaggerated for clarity, illustration, and convenience.

### DETAILED DESCRIPTION

The following detailed description is provided to assist the reader in gaining a comprehensive understanding of the methods, apparatuses, and/or systems described herein. However, various changes, modifications, and equivalents of the methods, apparatuses, and/or systems described herein will be apparent after an understanding of the disclosure of this application. For example, the sequences within and/or of operations described herein are merely examples, and are not limited to those set forth herein, but may be changed as will be apparent after an understanding of the disclosure of this application, except for sequences within and/or of operations necessarily occurring in a certain order. As another example, the sequences of and/or within operations may be performed in parallel, except for at least a portion of sequences of and/or within operations necessarily occurring in an order, e.g., a certain order. Also, descriptions of features that are known after an understanding of the disclosure of this application may be omitted for increased clarity and conciseness.

As used herein, the term “and/or” includes any one and any combination of any two or more of the associated listed items. The phrases “at least one of A, B, and C”, “at least one of A, B, or C”, and the like are intended to have disjunctive meanings, and these phrases “at least one of A, B, and C”, “at least one of A, B, or C”, and the like also include examples where there may be one or more of each of A, B, and/or C (e.g., any combination of one or more of each of A, B, and C), unless the corresponding description and embodiment necessitates such listings (e.g., “at least one of A, B, and C”) to be interpreted to have a conjunctive meaning.

Throughout the specification, when a component or element is described as “on,” “connected to,” “coupled to,” or “joined to” another component, element, or layer, it may be directly (e.g., in contact with the other component, element, or layer) “on,” “connected to,” “coupled to,” or “joined to” the other component element, or layer, or there may reasonably be one or more other components elements, or layers intervening therebetween. When a component or element is described as “directly on”, “directly connected to,” “directly coupled to,” or “directly joined to” another component element, or layer, there can be no other components, elements, or layers intervening therebetween. Likewise, expressions, for example, “between” and “immediately between” and “adjacent to” and “immediately adjacent to” may also be construed as described in the foregoing.

The terminology used herein is for describing various examples only and is not to be used to limit the disclosure. The articles “a,” “an,” and “the” are intended to include the plural forms as well, unless the context clearly indicates otherwise. As non-limiting examples, terms “comprise” or “comprises,” “include” or “includes,” and “have” or “has” specify the presence of stated features, numbers, operations, members, elements, and/or combinations thereof, but do not preclude the presence or addition of one or more other features, numbers, operations, members, elements, and/or combinations thereof, or the alternate presence of an alternative stated features, numbers, operations, members, elements, and/or combinations thereof. Additionally, while one embodiment may set forth such terms “comprise” or “comprises,” “include” or “includes,” and “have” or “has” specify the presence of stated features, numbers, operations, members, elements, and/or combinations thereof, other embodiments may exist where one or more of the stated features, numbers, operations, members, elements, and/or combinations thereof are not present.

Unless otherwise defined, all terms used herein including technical and scientific terms have the same meanings as those commonly understood by one of ordinary skill in the art to which this disclosure pertains and based on an understanding of the disclosure of the present application. Terms such as those defined in commonly used dictionaries are to be interpreted as having a meaning that is consistent with their meaning in the context of the relevant art and the disclosure of the present application, and are not to be interpreted in an idealized or overly formal sense unless expressly so defined herein.

Although terms such as “first,” “second,” and “third”, or A, B, (a), (b), and the like may be used herein to describe various members, components, regions, layers, or sections, these members, components, regions, layers, or sections are not to be limited by these terms. Each of these terminologies is not used to define an essence, order, or sequence of corresponding members, components, regions, layers, or sections, for example, but used merely to distinguish the corresponding members, components, regions, layers, or sections from other members, components, regions, layers, or sections. Thus, a first member, component, region, layer, or section referred to in the examples described herein may also be referred to as a second member, component, region, layer, or section without departing from the teachings of the examples.

The features described herein may be embodied in different forms, and are not to be construed as being limited to the examples described herein. Rather, the examples described herein have been provided merely to illustrate some of the many possible ways of implementing the methods, apparatuses, and/or systems described herein that will be apparent after an understanding of the disclosure of this application. The use of the term “may” herein with respect to an example or embodiment (e.g., as to what an example or embodiment may include or implement) means that at least one example or embodiment exists where such a feature is included or implemented, while all examples are not limited thereto. The use of the terms “example” or “embodiment” herein have a same meaning (e.g., the phrasing “in one example” has a same meaning as “in one embodiment”, and “one or more examples” has a same meaning as “in one or more embodiments”).

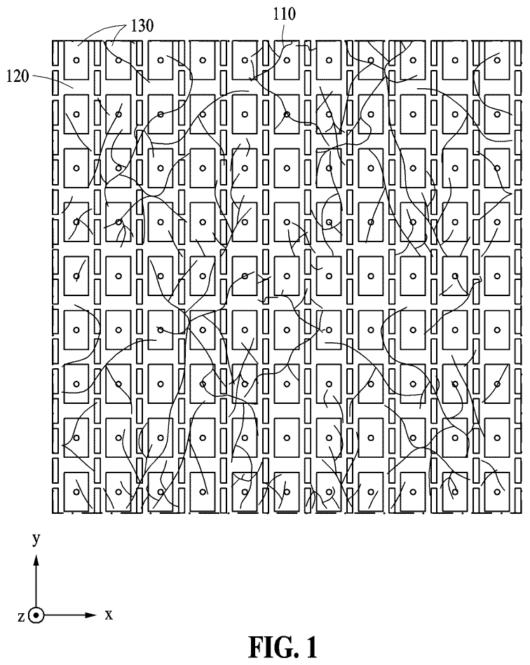

Hereinafter, the examples are described in detail with reference to the accompanying drawings. When describing the examples with reference to the accompanying drawings, like reference numerals refer to like components and a repeated description related thereto is omitted. FIG. 1 illustrates an example of a device for measuring an electrical signal of a cell.

Referring to FIG. 1, the device for measuring an electrical signal of a cell may detect an electrical signal generated by a cell 110. For example, the cell 110 may be a neuron but is not limited thereto. A plurality of cells 110 may send and receive an electrical signal between each other. The device may be provided immersed in an aqueous solution. The electrical signal generated by the cell 110 may be transmitted to an electrode through the aqueous solution.

The device may include a system base 120 and an electrode 130 for receiving the electrical signal of the cell 110. The electrode 130 may be provided in plurality. The electrode 130 may sit on the system base 120. The system base 120 may include a circuit that collects and analyzes signals detected by the electrode 130. The system base 120 and electrode 130 may be electrically and physically connected.

The electrode 130 may be arranged in plurality on a planar surface. A plurality of electrodes 130 may be provided horizontally and vertically aligned. The plurality of electrodes 130 may support the plurality of cells 110 and may each detect an electrical signal generated by the cells 110. The plurality of electrodes 130 may be disposed side by side on an XY plane. The electrical signal generated by the cell 110 may move in the Z-axis direction through the aqueous solution and be transmitted to the inside of the electrode 130. The plurality of cells 110 may be cultured or mounted on the plurality of electrodes 130 so that an electrical signal generated at the cellular level may be sensed through the electrodes 130.

The electrodes 130 may be for stimulating and sensing the cells 110 and may be used to sense an electrical signal at the cellular level. The device may measure or determine a state of a cell through a contact impedance that occurs between the electrodes and the cells.

The electrodes 130 described above may be implemented as microelectrode array (MEA). For example, the MEA may include tens of thousands of channels within a chip to analyze connections between neurons or brain cells at the network level.

A medium for culturing a cell may include a nutrient source in the form of a liquid or a gel conceived to proliferate a microorganism, a cell, and the like. The medium may also be referred to as a ‘culture soil’ or a ‘culture medium’. The medium may be contained in a cell culture chamber or a cell culture vessel. The medium may be, for example, a saline solution, but is not limited thereto.

The device may be used for a drug screening system to test the efficacy or toxicity of drugs and/or for extracting memory information from neurons by sensing an electrical signal at the cellular level and analyzing a response status of a cell to drugs or a mechanism of neuronal operation.

For the analysis of a large-scale biological neural network, the device may sense an electrical signal from neurons using the MEA and may obtain information on a relationship between cells. The device may perform intracellular recording, which is an operation of a highly sensitive sensing of an intracellular potential through an electroporation technique in which an electric current is applied to an electrode to form a hole in a membrane of a cell adjacent to the electrode. The device may also perform extracellular recording, which is an operation of sensing an intracellular potential when no electroporation has been generated in a cell.

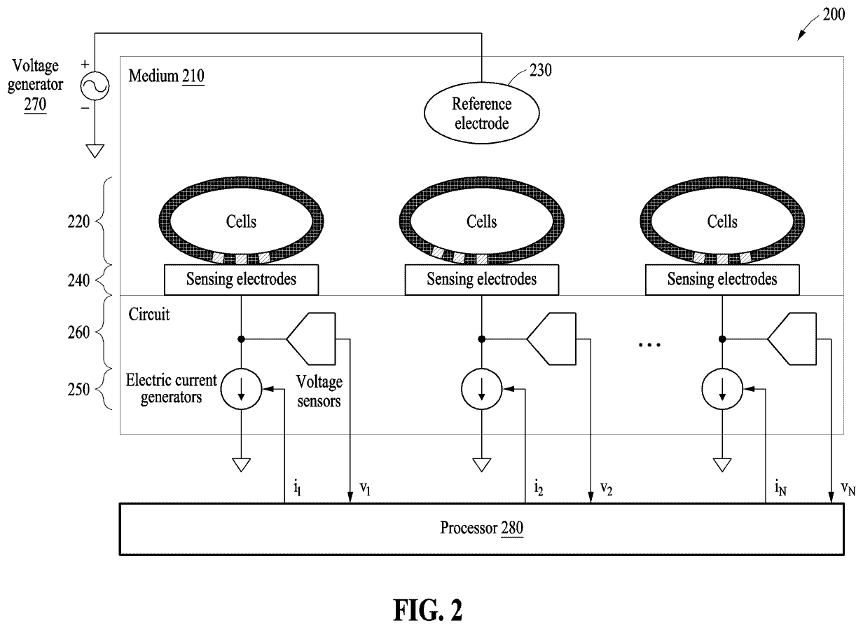

FIG. 2 illustrates an example of components of a device for measuring a potential of a cell.

Referring to FIG. 2, an electronic device 200 may include a reference electrode 230, a plurality of sensing electrodes 240, a plurality of electric current generators 250, a plurality of voltage sensors 260, a voltage generator 270, and a processor 280 (e.g., one or more processors). Cells 220 may be cultured in a medium 210, and the plurality of sensing electrodes 240 may come into contact with the medium 210 for culturing the cells 220.

A cell may be cultured on each of the plurality of sensing electrodes 240, and a location or shape of the cell cultured on each sensing electrode may vary. For example, a cell may be cultured on a sensing electrode in close contact, whereas another cell may not be in close contact with another sensing electrode but be cultured spaced apart by a certain size or at an angle on the other sensing electrode.

Electroporation, which forms a hole in a cell membrane, may be implemented by applying an electric current to a cell through a sensing electrode on which the cell is cultured. To this end, on each sensing electrode, an electric current generator for applying an electric current to the sensing electrode and a voltage sensor for measuring a voltage formed on the sensing electrode may be disposed. When an electric current generator applies an electric current to a corresponding sensing electrode, all or a portion of the applied electric current may flow through a cell cultured on the sensing electrode. Thus, for ease of description, the expression “applying an electric current to a sensing electrode” and the expression “applying an electric current to a cell” may be interchangeably used herein. In addition, since it is self-evident that electroporation occurs in a cell due to an electric current applied to the cell, the expression “electroporation occurs in a cell” and the expression “electroporation occurs in a sensing electrode” may be interchangeably used herein for ease of description.

Each voltage sensor 260 may include an analog front-end (AFE). The AFE may stimulate a cell 220 that comes into contact with or is adjacent to a corresponding sensing electrode 240 and may record an excitation of the cell 220 by stimulation in the form of an electrical signal. The AFE may be a component for digitizing an analog signal in a digital system for processing an analog signal into a digital signal and may be a circuit in which an analog preposition circuit and an analog-to-digital converter (ADC) are integrated in a single chip. An analog voltage V of an input terminal connected to the corresponding sensing electrode 240 of the AFE and a digital value D of an output terminal connected to the processor 280 may have a relationship of V=G. D. Here, G denotes a gain of the AFE, and * denotes multiplication.

The AFE may include, for example, a stimulator for stimulating a cell, a signal amplifier that amplifies a signal detected in cells, and an ADC that converts an amplified (analog) signal to a digital signal, but is not limited thereto. Alternatively, the AFE may include, for example, an instrumentation amplifier (IA) and an ADC corresponding to each sensing electrode 240. Some AFEs may amplify an electrical signal measured by each sensing electrode 240 through a corresponding IA and may convert the amplified electrical signal to a digital signal through a corresponding ADC.

The reference electrode 230 may be connected to a ground and disposed in the medium 210. The voltage generator 270 may be disposed between the reference electrode 230 and a ground and may generate a predetermined reference voltage. Here, the reference voltage may represent an alternating current (AC) component value, e.g., a component value that changes rapidly over time. When an electric current is applied by an electric current generator 250 corresponding to each of the plurality of sensing electrodes 240, the electric current generated by the electric current generator 250 may flow along a closed loop formed by the voltage generator 270, the reference electrode 230, each sensing electrode 240, and each electric current generator 250.

The plurality of electric current generators 250 may operate under the control by the processor 280. In addition, information on a voltage sensed by each of the plurality of voltage sensors 260 may be transmitted to the processor 280. The processor 280 may function as both a controller that controls the plurality of electric current generators 250 and a processor that processes information on the sensed voltage but may be referred to as a processor herein for ease of description.

For a highly sensitive sensing of an electrical signal from a very small cell, a magnitude of an impedance of a sensing electrode may be kept small to minimize loss of a signal (e.g., voltage) generated through the sensing electrode. The impedance of the sensing electrode may be determined by a ratio of the voltage to the current (i.e., V/I) applied to the sensing electrode, but a parasitic element of the sensing electrode and the gain of a voltage sensor may affect the impedance. Since a size of the parasitic element may vary depending on a position of each electrode of multiple sensing electrodes and a gain deviation of the voltage sensor may exist for each electrode due to a process deviation, the electronic device of one or more embodiments may determine the impedance of the sensing electrode regardless of the parasitic element and the gain of the voltage sensor, thereby more accurately determining the impedance.

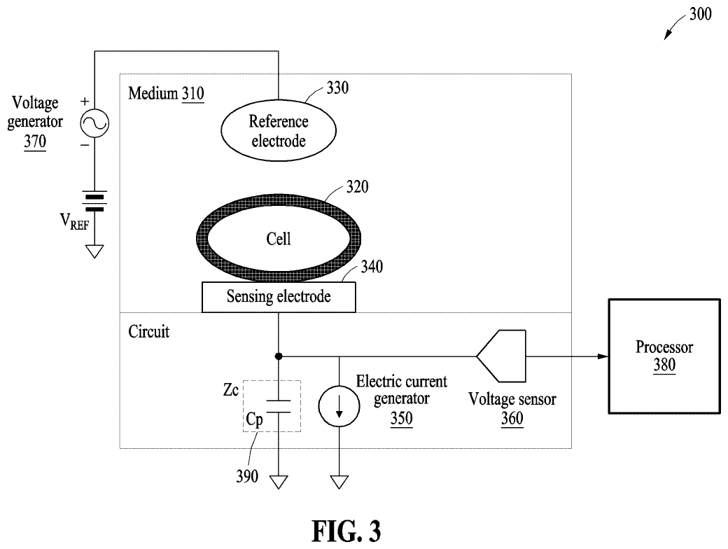

FIG. 3 illustrates an operation of elements in a device based on a target sensing electrode.

Referring to FIG. 3, an electronic device 300 may include a reference electrode 330, a sensing electrode 340, an electric current generator 350, a voltage sensor 360, a voltage generator 370, and a processor 380 (e.g., one or more processors). A voltage source VREF connected in series to the voltage generator 370 may indicate a DC voltage, and a DC element may not be considered in a subsequent operation of determining an impedance of the sensing electrode 340. FIG. 3 may illustrate an operation of elements in the device 300 based on a target sensing electrode, which may be one of a plurality of sensing electrodes.

An impedance size of the sensing electrode 340 may be kept small in order to accurately sense an electrical signal of a cell 320. When a magnitude of the electrical signal of the cell 320 is very small, a parasitic element 390 of the sensing electrode 340 may affect the impedance. The parasitic element 390 may not be a circuit element that is physically present in the device 300, but may be generated by an AC signal (e.g., an AC electric current or an AC voltage) generated from the electric current generator 350 and/or the voltage generator 370. For example, the parasitic element 390 may be expressed as a parasitic impedance Z_{c} including a parasitic capacitance Cₚ.

The device 300 may determine in advance a level of accuracy with which the electrical signal may be sensed, by checking (e.g., measuring or determining) the impedance of the sensing electrode 340 when sensing the electrical signal of the cell 320. When the impedance of the sensing electrode 340 is greater than or equal to a predetermined reference value and accordingly a quality of the electrical signal being sensed is not expected to be satisfactory, the electrical signal may be sensed with the other sensing electrodes having an impedance less than the predetermined reference value, by lowering the impedance of the sensing electrode 340 or excluding the corresponding sensing electrode 340.

When a medium 310 is present, the reference electrode 330 and the sensing electrode 340 may be electrically connected to each other, and a closed loop may be formed by the voltage generator 370, the reference electrode 330, the sensing electrode 340, the electric current generator 350, and the parasitic element 390. On the contrary, when the medium 310 is removed, the reference electrode 330 and the sensing electrode 340 may not be electrically connected, and a closed loop may be formed by the sensing electrode 340, the electric current generator 350, and the parasitic element 390.

Hereinafter, an operation of measuring the impedance of the sensing electrode 340 is described in detail. The operation may be measuring the impedance of the sensing electrode 340 without considering the gain of the voltage sensor 360 and the parasitic element 390 that may vary for each electrode, by additionally using a voltage of the sensing electrode 340 when a reference voltage is applied to reference electrode 330 by the voltage generator 370 connected to the reference electrode 330.

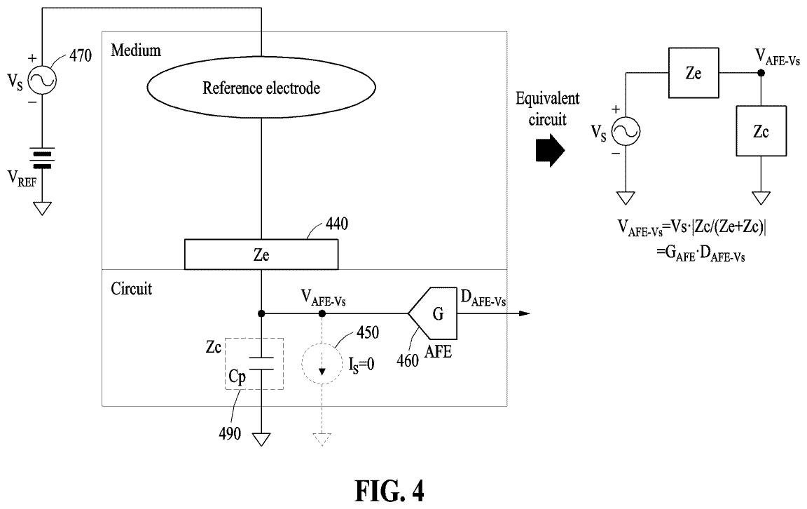

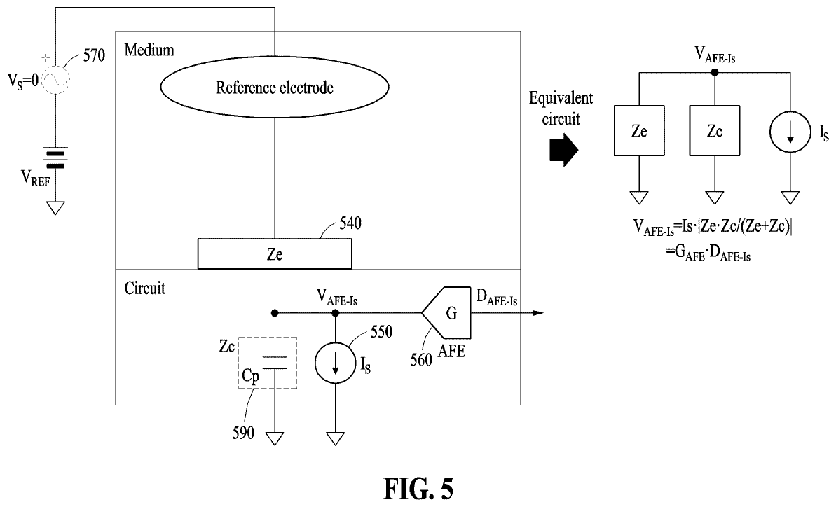

FIG. 4 and FIG. 5 each illustrate an example of an operation of measuring an impedance of a sensing electrode.

Referring to FIG. 4, in a state in which a medium is present, an electric current generator 450 may be turned off, and a reference voltage Vs may be applied by a voltage generator 470 shown on the left, and a corresponding equivalent circuit is shown on the right. When the electric current generator 450 is turned off and a reference electric current Iₛ is “0,” an electric current may not flow through a wire connected to the electric current generator 450 and accordingly the wire to the electric current generator 450 may be in an open state. An impedance of a sensing electrode 440 may be expressed as Zₑ, and an impedance of a parasitic element 490 may be expressed as Z_{c}.

Equation 1 below, for example, may be derived from a relationship between an input terminal and an output terminal of a voltage sensor 460 and the equivalent circuit.

$$\begin{matrix}{V}_{AFE - Vs} = Vs \cdot \vert Zc / ( Ze + Zc ) \vert \\ = {G}_{AFE} \cdot {D}_{AFE - Vs}\end{matrix} \qquad Equation 1$$

In Equation 1 above, V_{AFE-Vs} denotes a voltage of the sensing electrode 440 and may correspond to a voltage at the input terminal of the voltage sensor 460. Vₛ may denote the reference voltage Vₛ generated by the voltage generator 470, G_{AFE} may denote a gain of the voltage sensor 460, and D_{AFE-Vs} may denote a digital value output from the voltage sensor 460.

Referring to FIG. 5, in a state in which a medium is present, a voltage generator 570 may be turned off, and a reference electric current Iₛ may be applied by an electric current generator 550 shown on the left, and a corresponding equivalent circuit is shown on the right. When the voltage generator 570 is turned off and the reference voltage Vₛ is “0,” both ends of the voltage generator 570 may be shorted.

Equation 2 below, for example, may be derived from a relationship between an input terminal and an output terminal of a voltage sensor 560 and the equivalent circuit.

$$\begin{matrix}{V}_{AFE - Is} = Is \cdot \vert Ze \cdot Zc / ( Ze + Zc ) \vert \\ = {G}_{AFE} \cdot {D}_{AFE - Is}\end{matrix} \qquad Equation 2$$

In Equation 2 above, V_{AFE-Is} denotes a voltage of a sensing electrode 540 and may correspond to a voltage at the input terminal of the voltage sensor 560. Iₛ may denote the reference electric current Iₛ generated by the electric current generator 550, and D_{AFE-Is} may denote the digital value D_{AFE-Is} output from the voltage sensor 560.

When dividing the both sides of Equation 2 excluding V_{AFE-Is} by the both sides of Equation 1 excluding V_{AFE-Vs} and summarizing by an impedance Zₑ of the sensing electrode 540, Equation 3 below, for example, may be obtained.

$$\vert Ze \vert = {D}_{AFE - Is} / {D}_{AFE - Vs} \cdot Vs / Is \qquad Equation 3$$

In other words, the impedance Zₑ of the sensing electrode 540 may be determined based on the reference electric current Iₛ, the digital value D_{AFE-Is} output from the voltage sensor 560 when the reference electric current Iₛ is applied, the reference voltage Vₛ, and the digital value D_{AFE-Vs} output from the voltage sensor 460 when the reference voltage Vₛ is applied. Accordingly, an electronic device of one or more embodiments may determine the impedance Zₑ of the sensing electrode 540 regardless of a gain of the voltage sensor 560 or a parasitic element 590 of the sensing electrode 540, of which a size may vary for each electrode due to a position of an electrode disposition or a process deviation, thereby more accurately determining the impedance Zₑ.

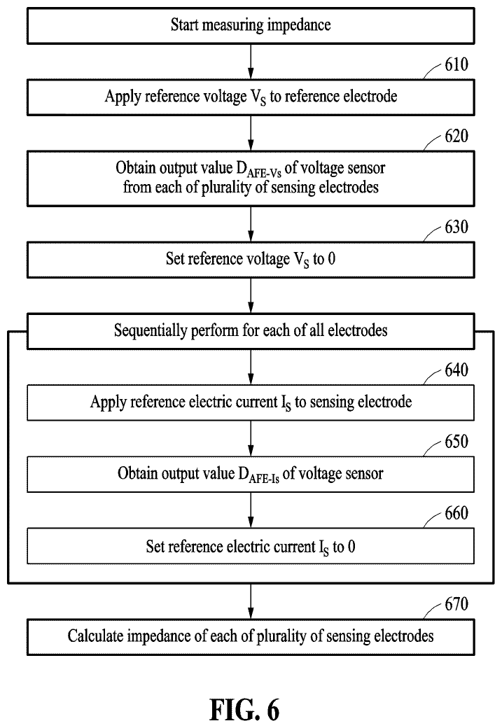

FIG. 6 illustrates a flowchart of an example of an operation of measuring an impedance of a sensing electrode.

In the following example, operations 610 to 670 may be performed sequentially in the order and manner as shown and described below with reference to FIG. 6, but the order of one or more of the operations may be changed, one or more of the operations may be omitted, and two or more of the operations may be performed in parallel or simultaneously without departing from the spirit and scope of the example embodiments described herein. Operations 610 to 670 may be performed by a device (e.g., the device 200 of FIG. 2 and/or the device 300 of FIG. 3).

In operation 610, the device may apply a reference voltage Vₛ to a reference electrode through a voltage generator. For example, the reference voltage Vₛ may be applied as a sine wave of a predetermined magnitude, but is not limited to the example described above.

In operation 620, the device may obtain the output value D_{AFE-Vs} of a voltage sensor from each of a plurality of sensing electrodes. For example, the output value D_{AFE-vs} may have a sine-wave shape corresponding to the reference voltage Vₛ and may be stored in a storage device.

In operation 630, the device may turn off the voltage generator to make the reference voltage Vₛ applied to the reference electrode “0.”

Subsequent operations 640 to 660 may be performed sequentially for each of the plurality of sensing electrodes. Operations 640 to 660 are described based on a target sensing electrode, which may be one of the plurality of sensing electrodes.

In operation 640, the device may apply the reference electric current Iₛ through a target electric current generator connected to the target sensing electrode. For example, the reference electric current Iₛ may be applied as a sine wave of a predetermined magnitude, but is not limited to the example described above.

In operation 650, the device may obtain an output value D_{AFE-Is} of a target voltage sensor connected to the target sensing electrode. For example, the output value D_{AFE-Is} may have a sine-wave shape corresponding to the reference electric current Iₛ and may be stored in the storage device.

In operation 660, the device may turn off the target electric current generator to make the reference electric current Iₛ applied to the sensing electrode “0.”

In operation 670, the device may determine the impedance Zₑ of each of the plurality of sensing electrodes according to the reference electric current Iₛ, the output value D_{AFE-Is} when the reference electric current Iₛ is applied, the reference voltage Vₛ, and the output value D_{AFE-Vs} when the reference voltage Vₛ is applied.

As the description above with reference to FIGS. 1 to 5 may be applied to each of the operations shown in FIG. 6, a more detailed description is omitted herein.

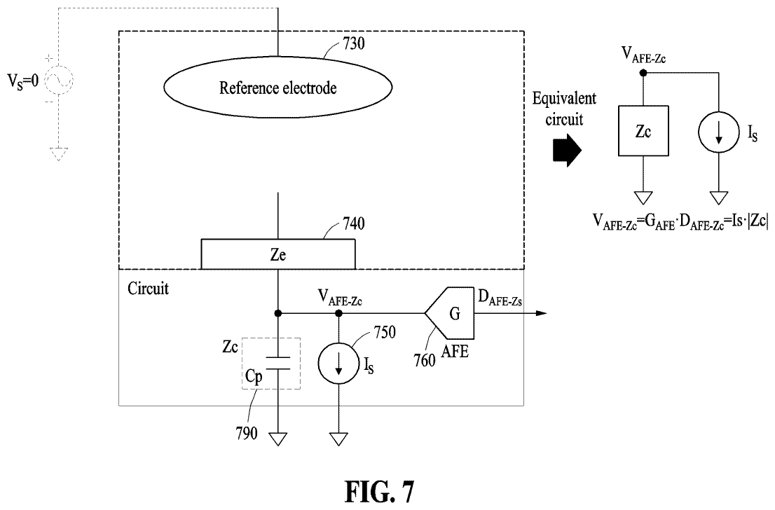

FIG. 7 illustrates an example of an operation of AFE calibration.

AFE calibration may refer to obtaining the gain G in V=G*D, which represents a relationship between the analog voltage V of an input terminal of a voltage sensor and the digital value D of an output terminal of the voltage sensor.

Referring to FIG. 7, a state for obtaining an additional mathematical formula for AFE calibration is shown, that is, a state in which a reference electrode 730 and a sensing electrode 740 are open to each other as no medium is present, and in which the reference electric current Iₛ is applied by an electric current generator 750.

When the reference electrode 730 and the sensing electrode 740 are open, the reference electric current Iₛ may flow to a parasitic component 790 of the sensing electrode 740, and based on this, an equivalent circuit may be as shown on the right side of FIG. 7.

Equation 4 below, for example, may be derived from a relationship between an input terminal and an output terminal of a voltage sensor 760 and the equivalent circuit.

$${V}_{AFE - Zc} = {G}_{AFE} \cdot {D}_{AFE - Zc} = Is \cdot \vert Zc \vert \qquad Equation 4$$

In Equation 4 above, V_{AFE-Zc} denotes a voltage of the sensing electrode 740 and may correspond to a voltage at the input terminal of the voltage sensor 760, Iₛ may denote the reference electric current generated by the current generator 750, G_{AFE} may denote a gain of the voltage sensor 760, and D_{AFE-Zc} may denote a digital value output from the voltage sensor 760.

When dividing the both sides of Equation 4 excluding V_{AFE-Zc} by the both sides of Equation 2 excluding V_{AFE-Is} and summarizing by the parasitic element Z. of the sensing electrode 740, Equation 5 below, for example, may be obtained.

$$\vert Zc \vert = \vert Ze \vert \cdot ( {D}_{AFE - Zc} / {D}_{AFE - Is} - 1 ) \qquad Equation 5$$

By substituting Equation 5 into both sides of Equation 1 with V_{AFE-Vs} excluded, Equation 6 below, for example, for obtaining the gain G_{AFE} of the voltage sensor 760 may be derived as follows.

$${G}_{AFE} = ( 1 - {D}_{AFE - Is} / {D}_{AFE - Zc} ) \cdot Vs / {D}_{AFE - Vs} \qquad Equation 6$$

In other words, the gain G_{AFE} of the voltage sensor 760 may be determined based on the reference voltage Vₛ, the digital value D_{AFE-Vs} output from the voltage sensor 460 when the reference voltage Vₛ is applied, and the digital values D_{AFE-Is} and D_{AFE-Zc} output from the voltage sensor 560 when the reference electric current Iₛ is applied. Accordingly, the electronic device of one or more embodiments may determine the gain G_{AFE} of the voltage sensor 760 regardless of the parasitic element 790 of the sensing electrode 740, of which a size may vary for each electrode due to a position of an electrode disposition or a process deviation, thereby more accurately determining the gain G_{AFE}.

AFE calibration may be performed by performing an operation of obtaining an output value of the voltage sensor 760 in a state shown in FIG. 7 in addition to the operation of measuring an impedance of the sensing electrode 740.

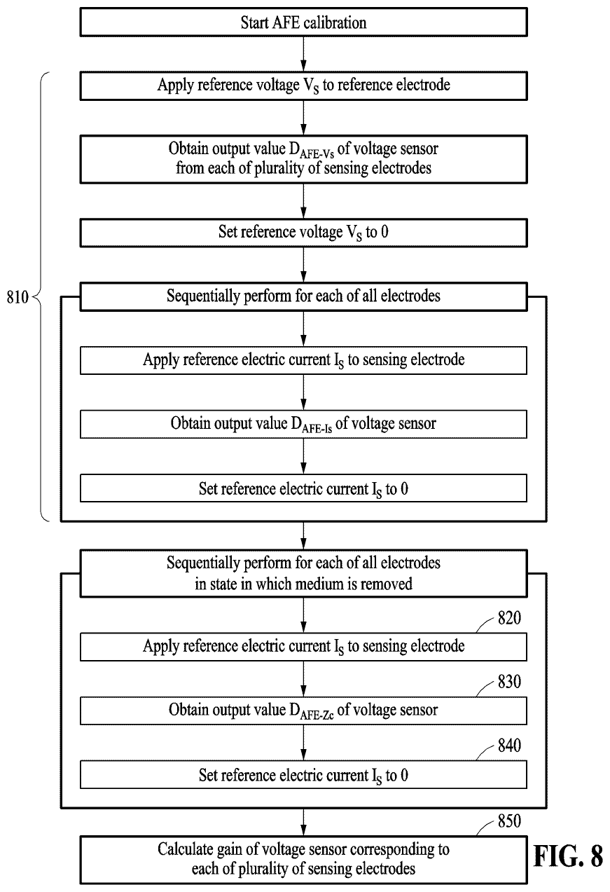

FIG. 8 illustrates a flowchart of an example of an operation of AFE calibration.

In the following example, operations 810 to 850 may be performed sequentially in the order and manner as shown and described below with reference to FIG. 8, but the order of one or more of the operations may be changed, one or more of the operations may be omitted, and two or more of the operations may be performed in parallel or simultaneously without departing from the spirit and scope of the example embodiments described herein. Operations 810 to 850 may be performed by a device (e.g., the device 200 of FIG. 2 and/or the device 300 of FIG. 3).

As operation 810 may be the same as operations 610 to 660 described above with reference to FIG. 6, a more detailed description is omitted herein.

Operations 820 to 840 to be subsequently performed may be performed sequentially for each of a plurality of electrodes in a state in which a medium is removed. Operations 820 to 840 are described based on a target sensing electrode, which may be one of a plurality of sensing electrodes.

In operation 820, the device may apply the reference electric current Iₛ through a target electric current generator connected to the target sensing electrode. For example, the reference electric current Iₛ may be applied as a sine wave of a predetermined magnitude, but is not limited to the example described above.

In operation 830, the device may obtain an output value D_{AFE-Zc} of a target voltage sensor connected to the target sensing electrode. For example, the output value D_{AFE-Zc} may have a sine-wave shape corresponding to the reference electric current Iₛ and may be stored in a storage device.

In operation 840, the device may turn off the target electric current generator to make the reference electric current Iₛ applied to the sensing electrode “0.”

In operation 850, the device may determine a gain of a voltage sensor corresponding to each of the plurality of sensing electrodes according to the reference voltage Vₛ, the output value D_{AFE-Vs} when the reference voltage Vₛ is applied, and the output values D_{AFE-Is} and D_{AFE-Zc} when the reference electric current Iₛ is applied.

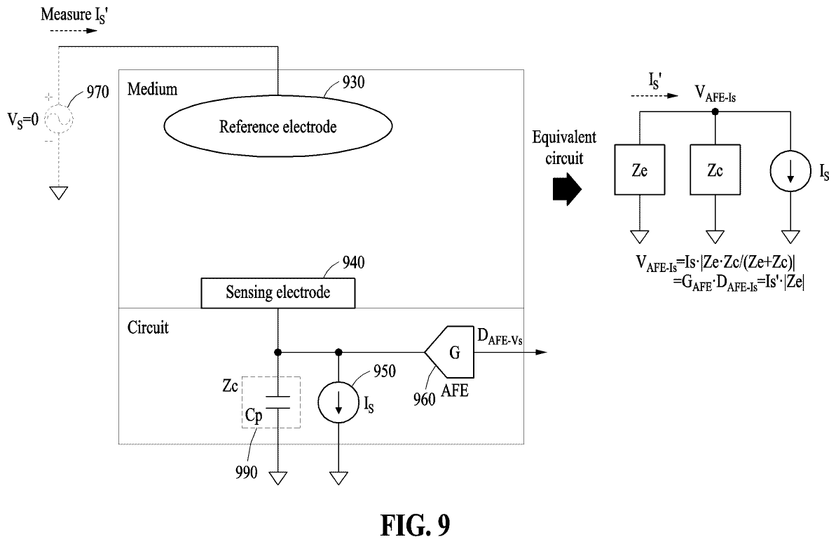

FIG. 9 illustrates an example of an operation of measuring an impedance of a sensing electrode based on a second reference electric current flowing through a reference electrode when a magnitude of a reference electric current generated by an electric current generator is uncertain.

Depending on an example, a magnitude of the reference electric current Iₛ generated by the electric current generator may be uncertain. When the magnitude of the reference electric current Iₛ is used when obtaining the impedance Zₑ of a sensing electrode 940 according to Equation 3 or obtaining the gain G_{AFE} of a voltage sensor 960 according to Equation 6, when the magnitude of the reference electric current Iₛ is uncertain, the impedance Zₑ or the gain G_{AFE} may also be uncertain. To prevent this, the electronic device of one or more embodiments may determine the impedance Zₑ or the gain G_{AFE} based on a second reference electric current flowing through a reference electrode 930.

Referring to FIG. 9, a state in which a medium is present, a voltage generator 970 may be turned off, and the reference electric current Iₛ may be applied by an electric current generator 950 shown on the left, and a corresponding equivalent circuit is shown on the right.

When a portion of the reference electric current Iₛ generated by the electric current generator 950 flows to a parasitic element 990 of the sensing electrode 940, a magnitude of the second reference electric current Iₛ' flowing through the reference electrode 930 may be less than the reference electric current Iₛ.

Equation 7 below, for example, may be derived from a relationship between an input terminal and an output terminal of the voltage sensor 960 and the equivalent circuit.

$$\begin{matrix}{V}_{AFE - Is} = Is \cdot \vert Ze \cdot Zc / ( Ze + Zc ) \vert \\ = {G}_{AFE} \cdot {D}_{AFE - Is} = Is ’ \cdot \vert Ze \vert\end{matrix} \qquad Equation 7$$

Using Equation 7 above, a relationship between the reference electric current Iₛ and the second reference electric current Iₛ' may be summarized as Equation 8 below, for example.

$$Is = Is ’ \cdot ( 1 + Ze / Zc ) \qquad Equation 8$$

Subsequently, as described with reference to FIG. 7, Equation 5 may be derived in a state in which the reference electric current Iₛ is applied by the electric current generator 750 after the medium is removed. By substituting Equation 5 into Equation 8, Equation 9 below, for example, for determining the reference electric current Iₛ based on the second reference electric current Iₛ' may be derived as follows.

$$Is {}_{}^{\cdot } {=}_{\cdot }^{} Is ’ / ( 1 - {D}_{AFE - Is} / {D}_{AFE - Zc} ) \qquad Equation 9$$

By substituting Equation 9 above into Equation 3, Equation 10 below, for example, for obtaining the impedance Zₑ of the sensing electrode 940 based on the second reference electric current Iₛ' may be derived as follows.

$$\vert Ze \vert = {D}_{AFE - Is} / {D}_{AFE - Vs} \cdot Vs / ( Is ’ / ( 1 - {D}_{AFE - Is} / {D}_{AFE - Zc} ) ) \qquad Equation 10$$

In other words, the impedance Zₑ of the sensing electrode 940 may be determined based on the second reference electric current Iₛ′, the digital value D_{AFE-Is} output from the voltage sensor 960 when the reference electric current Iₛ is applied, the reference voltage Vₛ, and the digital value D_{AFE-Vs} output from the voltage sensor 460 when the reference voltage Vₛ is applied. Accordingly, the electronic device of one or more embodiments may determine the impedance of the sensing electrode 940 regardless of not only a gain of the voltage sensor 960 and a parasitic element 990 of the sensing electrode 940, of which a size may vary for each electrode due to a position of an electrode disposition or a process deviation, but also the reference electric current Iₛ, of which a magnitude may be uncertain, thereby more accurately determining the impedance.

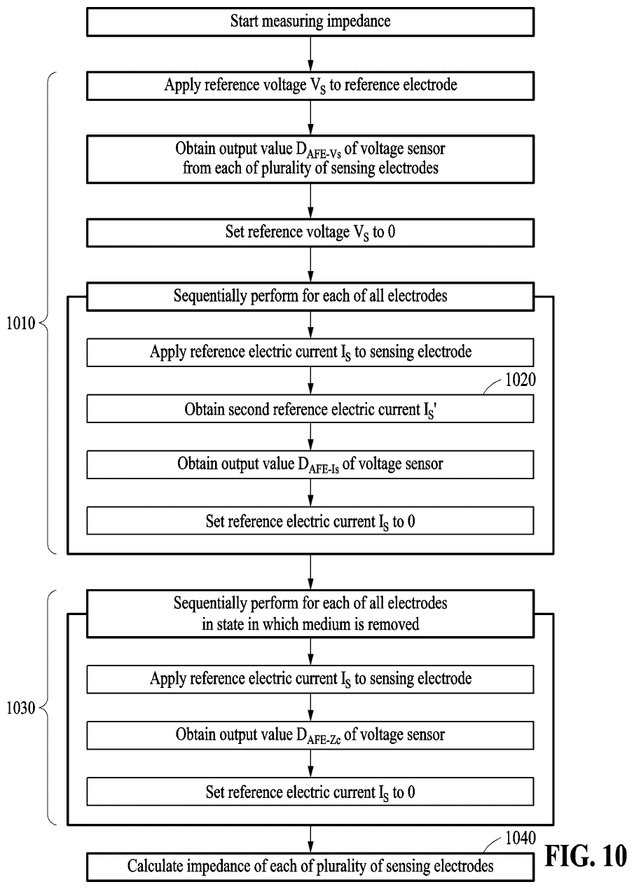

FIG. 10 illustrates a flowchart of an example of an operation of measuring an impedance of a sensing electrode based on a second reference electric current flowing through a reference electrode.

In the following example, operations 1010 to 1040 may be performed sequentially in the order and manner as shown and described below with reference to FIG. 10, but the order of one or more of the operations may be changed, one or more of the operations may be omitted, and two or more of the operations may be performed in parallel or simultaneously without departing from the spirit and scope of the example embodiments described herein. Operations 1010 to 1040 may be performed by a device (e.g., the device 200 of FIG. 2 and/or the device 300 of FIG. 3).

As operation 1010 excluding operation 1020 may be the same as operations 610 to 660 described above with reference to FIG. 6, a more detailed description is omitted herein.

In operation 1020, the device may obtain the second reference electric current Iₛ' flowing through the reference electrode while the reference electric current Iₛ is being applied by a target electric current generator connected to a target sensing electrode. For example, the second reference electric current Iₛ' may have a sine-wave shape corresponding to the reference electric current Iₛ and may be stored in a storage device.

As operation 1030 may be the same as operations 820 to 840 described above with reference to FIG. 8, a more detailed description is omitted herein.

In operation 1040, the device may determine the impedance Zₑ of each of a plurality of sensing electrodes according to the second reference electric current Iₛ′, the output values D_{AFE-I s} and D_{AFE-Zc}, the reference voltage Vₛ, and the output value D_{AFE-Vs} when the reference voltage Vₛ is applied.

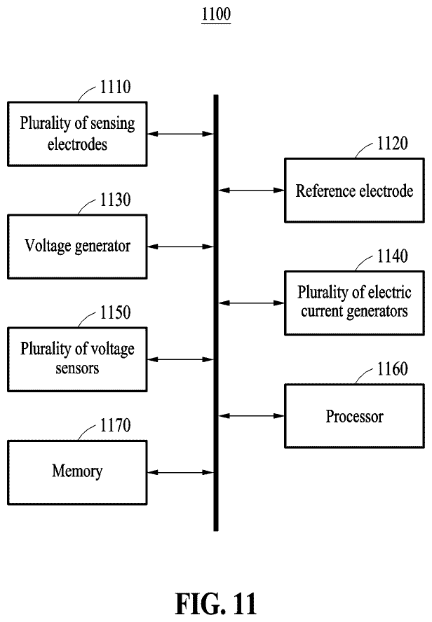

FIG. 11 illustrates an example of a device for measuring an electrical signal of a cell.

Referring to FIG. 11, an electronic device 1100 may include a plurality of sensing electrodes 1110, a reference electrode 1120, a voltage generator 1130, a plurality of electric current generators 1140, a plurality of voltage sensors 1150, a processor 1160 (e.g., one or more processors), and a memory 1170 (e.g., one or more memories).

The memory 1170 may include a non-transitory computer-readable storage medium storing instructions that, when executed by the processor 1160, configure the processor 1160 to perform any one, any combination, or all of operations and methods of the processor 1160 described herein. Further, the memory 1170 may include a storage device for storing output values of the plurality of voltage sensors 1150.

The plurality of sensing electrodes 1110 may come into contact with a medium for culturing cells. The plurality of sensing electrodes 1110 may be or include any one, any combination, or all of the sensing electrodes disclosed herein (e.g., the sensing electrodes 240, the sensing electrode 340, the sensing electrode 440, sensing electrode 540, the sensing electrode 740, and/or the sensing electrode 940).

The reference electrode 1120 may be connected to a ground and in contact with a medium. The reference electrode 1120 may be or include any one, any combination, or all of the reference electrodes disclosed herein (e.g., the reference electrode 230, the reference electrode 330, the reference electrode 730, and/or the reference electrode 930).

The voltage generator 1130 may be connected to the reference electrode 1120. The voltage generator 1130 may be or include any one, any combination, or all of the voltage generators disclosed herein (e.g., the voltage generator 270, the voltage generator 370, the voltage generator 470, the voltage generator 570, and/or the voltage generator 970).

Each of the plurality of electric current generators 1140 may be connected to each of the plurality of sensing electrodes 1110 and may apply an electric current to a corresponding sensing electrode. The electric current generators 1140 may be or include any one, any combination, or all of the electric current generators disclosed herein (e.g., the electric current generators 250, the electric current generator 350, the electric current generator 450, the electric current generator 550, the electric current generator 750, and/or the electric current generator 950).

Each of the plurality of voltage sensors 1150 may be connected to each of the plurality of sensing electrodes 1110 and may measure a voltage of a corresponding sensing electrode. The voltage sensors 1150 may be or include any one, any combination, or all of the voltage sensors disclosure herein (e.g., the voltage sensors 260, the voltage sensors 360, the voltage sensor 460, the voltage sensor 560, the voltage sensor 760, and/or the voltage sensor 960).

The processor 1160 may obtain a first value output from a target voltage sensor connected to a target sensing electrode among a plurality of sensing electrodes in response to applying a reference voltage by a voltage generator, may obtain a second value output from the target voltage sensor in response to applying a reference electric current by a target electric current generator connected to the target sensing electrode among a plurality of electric current generators, and may determine an impedance of the target sensing electrode based on the reference voltage, the reference electric current, the first value, and the second value. The processor 1160 may be or include any one, any combination, or all of the processors disclosed herein (e.g., the processor 280 and/or the processor 380).

The device 1100 may process any one, any combination, or all of the operations and/or methods described herein with reference to FIGS. 1-10 and 12, and a more detailed description thereof is omitted herein. The device 1100 may be or include any one, any combination, or all of the devices disclosed herein (e.g., the device 200 and/or the device 300).

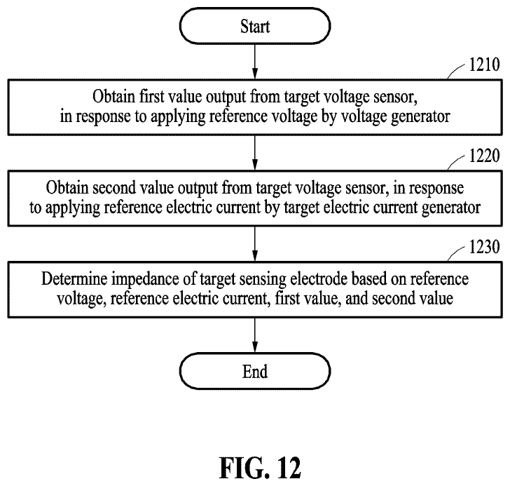

FIG. 12 illustrates an example of an operating method of a device for measuring an electrical signal of a cell.

In the following example, operations 1210 to 1230 may be performed sequentially in the order and manner as shown and described below with reference to FIG. 12, but the order of one or more of the operations may be changed, one or more of the operations may be omitted, and two or more of the operations may be performed in parallel or simultaneously without departing from the spirit and scope of the example embodiments described herein. Operations 1210 to 1230, which may be performed after electroporation is initiated, may be performed by at least one component (e.g., a processor) of a device (e.g., the device 200 of FIG. 2, the device 300 of FIG. 3, and/or the device 1100 of FIG. 11).

In operation 1210, the device may, in response to applying a reference voltage by a voltage generator connected to a ground and a reference electrode in contact with a medium for culturing cells, obtain a first value output from a target voltage sensor connected to a target sensing electrode among a plurality of sensing electrodes in contact with the medium.

In operation 1220, the device may obtain a second value output from the target voltage sensor in response to applying a reference electric current by a target electric current generator connected to the target sensing electrode.

In operation 1230, the device may determine an impedance of the target sensing electrode based on the reference voltage, the reference electric current, the first value, and the second value.

As the description above with reference to FIGS. 1 to 11 may be applied to each of the operations shown in FIG. 12, a more detailed description is omitted herein.

The system bases, electrodes, devices, reference electrodes, sensing electrodes, electric current generators, voltage sensors, voltage generator, processors, parasitic elements, memories, system base 120, electrode 130, device 200, reference electrode 230, sensing electrodes 240, electric current generators 250, voltage sensors 260, voltage generator 270, processor 280, device 300, reference electrode 330, sensing electrode 340, electric current generator 350, voltage sensor 360, voltage generator 370, processor 380, parasitic element 390, sensing electrode 440, electric current generator 450, voltage sensor 460, voltage generator 470, parasitic element 490, sensing electrode 540, electric current generator 550, voltage sensor 560, voltage generator 570, parasitic element 590, reference electrode 730, sensing electrode 740, electric current generator 750, voltage sensor 760, parasitic component 790, reference electrode 930, sensing electrode 940, electric current generator 950, voltage sensor 960, voltage generator 970, parasitic element 990, device 1100, sensing electrodes 1110, reference electrode 1120, voltage generator 1130, electric current generators 1140, voltage sensors 1150, processor 1160, and memory 1170 described herein, including descriptions with respect to respect to FIGS. 1-12, are implemented by or representative of hardware components. As described above, or in addition to the descriptions above, examples of hardware components that may be used to perform the operations described in this application where appropriate include controllers, sensors, generators, drivers, memories, comparators, arithmetic logic units, adders, subtractors, multipliers, dividers, integrators, and any other electronic components configured to perform the operations described in this application. In other examples, one or more of the hardware components that perform the operations described in this application are implemented by computing hardware, for example, by one or more processors or computers. A processor or computer may be implemented by one or more processing elements, such as an array of logic gates, a controller and an arithmetic logic unit, a digital signal processor, a microcomputer, a programmable logic controller, a field-programmable gate array, a programmable logic array, a microprocessor, or any other device or combination of devices that is configured to respond to and execute instructions in a defined manner to achieve a desired result. In one example, a processor or computer includes, or is connected to, one or more memories storing instructions or software that are executed by the processor or computer. Hardware components implemented by a processor or computer may execute instructions or software, such as an operating system (OS) and one or more software applications that run on the OS, to perform the operations described in this application. The hardware components may also access, manipulate, process, create, and store data in response to execution of the instructions or software. For simplicity, the singular term “processor” or “computer” may be used in the description of the examples described in this application, but in other examples multiple processors or computers may be used, or a processor or computer may include multiple processing elements, or multiple types of processing elements, or both. For example, a single hardware component or two or more hardware components may be implemented by a single processor, or two or more processors, or a processor and a controller. One or more hardware components may be implemented by one or more processors, or a processor and a controller, and one or more other hardware components may be implemented by one or more other processors, or another processor and another controller. One or more processors, or a processor and a controller, may implement a single hardware component, or two or more hardware components. As described above, or in addition to the descriptions above, example hardware components may have any one or more of different processing configurations, examples of which include a single processor, independent processors, parallel processors, single-instruction single-data (SISD) multiprocessing, single-instruction multiple-data (SIMD) multiprocessing, multiple-instruction single-data (MISD) multiprocessing, and multiple-instruction multiple-data (MIMD) multiprocessing.

The methods illustrated in, and discussed with respect to, FIGS. 1-12 that perform the operations described in this application are performed by computing hardware, for example, by one or more processors or computers, implemented as described above implementing instructions (e.g., computer or processor/processing device readable instructions) or software to perform the operations described in this application that are performed by the methods. For example, a single operation or two or more operations may be performed by a single processor, or two or more processors, or a processor and a controller. One or more operations may be performed by one or more processors, or a processor and a controller, and one or more other operations may be performed by one or more other processors, or another processor and another controller. One or more processors, or a processor and a controller, may perform a single operation, or two or more operations.

Instructions or software to control computing hardware, for example, one or more processors or computers, to implement the hardware components and perform the methods as described above may be written as computer programs, code segments, instructions or any combination thereof, for individually or collectively instructing or configuring the one or more processors or computers to operate as a machine or special-purpose computer to perform the operations that are performed by the hardware components and the methods as described above. In one example, the instructions or software include machine code that is directly executed by the one or more processors or computers, such as machine code produced by a compiler. In another example, the instructions or software includes higher-level code that is executed by the one or more processors or computer using an interpreter. The instructions or software may be written using any programming language based on the block diagrams and the flow charts illustrated in the drawings and the corresponding descriptions herein, which disclose algorithms for performing the operations that are performed by the hardware components and the methods as described above.

The instructions or software to control computing hardware, for example, one or more processors or computers, to implement the hardware components and perform the methods as described above, and any associated data, data files, and data structures, may be recorded, stored, or fixed in or on one or more non-transitory computer-readable storage media, and thus, not a signal per se. As described above, or in addition to the descriptions above, examples of a non-transitory computer-readable storage medium include one or more of any of read-only memory (ROM), random-access programmable read only memory (PROM), electrically erasable programmable read-only memory (EEPROM), random-access memory (RAM), dynamic random access memory (DRAM), static random access memory (SRAM), flash memory, non-volatile memory, CD−ROMs, CD−Rs, CD+Rs, CD−RWs, CD+RWs, DVD−ROMs, DVD−Rs, DVD+Rs, DVD−RWs, DVD+RWs, DVD−RAMs, BD−ROMs, BD−Rs, BD−R LTHs, BD−REs, blue-ray or optical disk storage, hard disk drive (HDD), solid state drive (SSD), flash memory, a card type memory such as multimedia card micro or a card (for example, secure digital (SD) or extreme digital (XD)), magnetic tapes, floppy disks, magneto-optical data storage devices, optical data storage devices, hard disks, solid-state disks, and/or any other device that is configured to store the instructions or software and any associated data, data files, and data structures in a non-transitory manner and provide the instructions or software and any associated data, data files, and data structures to one or more processors or computers so that the one or more processors or computers can execute the instructions. In one example, the instructions or software and any associated data, data files, and data structures are distributed over network-coupled computer systems so that the instructions and software and any associated data, data files, and data structures are stored, accessed, and executed in a distributed fashion by the one or more processors or computers.

While this disclosure includes specific examples, it will be apparent after an understanding of the disclosure of this application that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner, and/or replaced or supplemented by other components or their equivalents.

Therefore, in addition to the above and all drawing disclosures, the scope of the disclosure is also inclusive of the claims and their equivalents, i.e., all variations within the scope of the claims and their equivalents are to be construed as being included in the disclosure.

What is claimed is:

1. An electronic device comprising:
a plurality of sensing electrodes in contact with a medium for culturing cells;
a reference electrode connected to a ground and in contact with the medium;
a voltage generator connected to the reference electrode;
a plurality of electric current generators, each of which is connected to each of the plurality of sensing electrodes and configured to apply an electric current to a corresponding one of the sensing electrodes;
a plurality of voltage sensors, each of which is connected to each of the plurality of sensing electrodes to measure the voltage of the corresponding sensing electrode; and
one or more processors configured to:
obtain, in response to applying a reference voltage by the voltage generator, a first value output from a target voltage sensor among the plurality of voltage sensors connected to a target sensing electrode among the plurality of sensing electrodes;
obtain, in response to applying a reference electric current by a target electric current generator connected to the target sensing electrode among the plurality of electric current generators, a second value output from the target voltage sensor; and
determine an impedance of the target sensing electrode based on the reference voltage, the reference electric current, the first value, and the second value.

2. The device of claim 1, wherein, for the determining of the impedance of the target sensing electrode, the one or more processors are further configured to determine the impedance of the target sensing electrode from the reference voltage, the reference electric current, the first value, and the second value, regardless of a parasitic element of the target sensing electrode and a gain of the target voltage sensor.

3. The device of claim 1, wherein the one or more processors are further configured to:
obtain a third value output from the target voltage sensor in response to applying the reference electric current by the target electric current generator after the medium is removed; and
determine a gain of the target voltage sensor based on the reference voltage, the first value, the second value, and the third value.

4. The device of claim 1, wherein the one or more processors are further configured to:
sense a second reference electric current flowing through the reference electrode in response to applying the reference electric current by the target electric current generator;
obtain a fourth value output from the target voltage sensor in response to applying a fourth reference electric current by the target electric current generator after the medium is removed; and
determine the impedance of the target sensing electrode based on the reference voltage, the second reference electric current, the first value, the second value, and the fourth value.

5. The device of claim 4, wherein the reference electric current applied by the target electric current generator is determined based on the second reference electric current, the second value, and the fourth value.

6. The device of claim 1, wherein,
in response to applying the reference voltage by the voltage generator, the plurality of electric current generators do not operate, and
in response to applying the reference electric current by one of the plurality of electric current generators, other electric current generators than the one and the voltage generator do not operate.

7. The device of claim 1, wherein
the reference voltage applied by the voltage generator is an alternating current (AC) voltage,
the reference electric current applied by the target electric current generator is an AC electric current, and
the target voltage sensor is configured to output a digital value amplified according to a predetermined gain of an analog value of a voltage detected at an input terminal of the target voltage sensor.

8. The device of claim 1, wherein each of the plurality of electric current generators and each of the plurality of voltage sensors are disposed connected to a sensing electrode that corresponds to an inside of the device.

9. A method of a device, the method comprising:
obtaining, in response to applying a reference voltage by a voltage generator connected to a ground and a reference electrode in contact with a medium for culturing cells, a first value output from a target voltage sensor connected to a target sensing electrode among a plurality of sensing electrodes in contact with the medium;
obtaining, in response to applying a reference electric current by a target electric current generator connected to the target sensing electrode, a second value output from the target voltage sensor; and
determining an impedance of the target sensing electrode based on the reference voltage, the reference electric current, the first value, and the second value.

10. The method of claim 9, wherein the determining of the impedance of the target sensing electrode comprises determining the impedance of the target sensing electrode from the reference voltage, the reference electric current, the first value, and the second value, regardless of a parasitic element of the target sensing electrode and a gain of the target voltage sensor.

11. The method of claim 9, further comprising:
obtaining a third value output from the target voltage sensor in response to applying the reference electric current by the target electric current generator after the medium is removed; and
determining a gain of the target voltage sensor based on the reference voltage, the first value, the second value, and the third value.

12. The method of claim 9, further comprising:
sensing a second reference electric current flowing through the reference electrode in response to applying the reference electric current by the target electric current generator;
obtaining a fourth value output from the target voltage sensor in response to applying a fourth reference electric current by the target electric current generator after the medium is removed; and
determining the impedance of the target sensing electrode based on the reference voltage, the second reference electric current, the first value, the second value, and the fourth value.

13. The method of claim 12, wherein the reference electric current applied by the target electric current generator is determined based on the second reference electric current, the second value, and the fourth value.

14. The method of claim 9, wherein,
in response to applying the reference voltage by the voltage generator, a plurality of electric current generators do not operate, and
in response to applying the reference electric current by one of the plurality of electric current generators, other electric current generators than the one and the voltage generator do not operate.

15. The method of claim 9, wherein
the reference voltage applied by the voltage generator is an alternating current (AC) voltage,
the reference electric current applied by the target electric current generator is an AC electric current, and
the target voltage sensor is configured to output a digital value amplified according to a predetermined gain of an analog value of a voltage detected at an input terminal.

16. The method of claim 9, wherein each of the plurality of electric current generators and each of the plurality of voltage sensors, which are included in the device, are disposed connected to a sensing electrode that corresponds to an inside of the device.

17. A non-transitory computer-readable storage medium storing instructions that, when executed by one or more processors, configure the one or more processors to perform the method of claim 9.

18. An electronic device comprising:
one or more processors configured to:
obtain, in response to a reference voltage being applied by a voltage generator connected to a ground and a reference electrode in contact with a medium for culturing cells, a first value output from a voltage sensor connected to a sensing electrode in contact with the medium;
obtain, in response to a reference electric current being applied by a electric current generator connected to the sensing electrode, a second value output from the voltage sensor; and
determine an impedance of the sensing electrode based on the reference voltage, the reference electric current, the first value, and the second value.

19. The device of claim 18, wherein
the electric current generator is in an open state when the first value is output from the voltage sensor, and
the voltage generator is shorted when the second value is output from the voltage sensor.

20. The device of claim 18, wherein the one or more processors are further configured to determine a state of a cell of the cells based on the determined impedance.
